SEQUENCE LISTING (45 sequences)

SEQ ID NO: 1 - RNA, 30 nt, synthetic construct
  source 1..30: mol_type = other RNA; note = shRNA1
ttaatacgacctctctgttggattttgaca

SEQ ID NO: 2 - RNA, 22 nt, synthetic construct
  source 1..22: mol_type = other RNA; note = shRNA2
ggttcgcaacttcacacagagt

SEQ ID NO: 3 - RNA, 22 nt, synthetic construct
  source 1..22: mol_type = other RNA; note = shRNA3
caggcgtttgttggttgattaa

SEQ ID NO: 4 - RNA, 30 nt, synthetic construct
  source 1..30: mol_type = other RNA; note = shRNA4
gttttacgacaacgatgttggtttaggaca

SEQ ID NO: 5 - RNA, 22 nt, synthetic construct
  source 1..22: mol_type = other RNA; note = shRNA5
ggttcggttgttatatacgata

SEQ ID NO: 6 - RNA, 22 nt, synthetic construct
  source 1..22: mol_type = other RNA; note = shRNA6
ggttcagagagtctcctattta

SEQ ID NO: 7 - RNA, 21 nt, synthetic construct
  source 1..21: mol_type = other RNA; note = shRNA7
gtggtattcttgctagttaca

SEQ ID NO: 8 - RNA, 21 nt, synthetic construct
  source 1..21: mol_type = other RNA; note = shRNA8
ggatttgtcttctacaatttg

SEQ ID NO: 9 - RNA, 21 nt, synthetic construct
  source 1..21: mol_type = other RNA; note = shRNA9
cgaacgctttcttattacaaa

SEQ ID NO: 10 - RNA, 21 nt, synthetic construct
  source 1..21: mol_type = other RNA; note = shRNA10
cactgttgctacatcacgaac

SEQ ID NO: 11 - RNA, 21 nt, synthetic construct
  source 1..21: mol_type = other RNA; note = shRNA11
cctagtaataggtttcctatt

SEQ ID NO: 12 - RNA, 21 nt, synthetic construct
  source 1..21: mol_type = other RNA; note = shRNA12
gcgtgtagcaggtgactcagg

SEQ ID NO: 13 - RNA, 21 nt, synthetic construct
  source 1..21: mol_type = other RNA; note = shRNA13
gaccgcttctagaaagtgaac

SEQ ID NO: 14 - RNA, 21 nt, synthetic construct
  source 1..21: mol_type = other RNA; note = shRNA14
caaggacctgcctaaagaaat

SEQ ID NO: 15 - RNA, 21 nt, synthetic construct
  source 1..21: mol_type = other RNA; note = shRNA15
atggatttgtcttctacaatt

SEQ ID NO: 16 - RNA, 21 nt, synthetic construct
  source 1..21: mol_type = other RNA; note = shRNA16
cgtagtcgcaacagttcaaga

SEQ ID NO: 17 - RNA, 21 nt, synthetic construct
  source 1..21: mol_type = other RNA; note = shRNA17
ggtggaccctcagattcaact

SEQ ID NO: 18 - RNA, 21 nt, synthetic construct
  source 1..21: mol_type = other RNA; note = shRNA18
ggttcaccgctctcactcaac

SEQ ID NO: 19 - RNA, 21 nt, synthetic construct
  source 1..21: mol_type = other RNA; note = shRNA19
cgggaacgtggttgacctaca

SEQ ID NO: 20 - RNA, 21 nt, synthetic construct
  source 1..21: mol_type = other RNA; note = shRNA20
gcttatgtgtcaacctatact

SEQ ID NO: 21 - RNA, 21 nt, synthetic construct
  source 1..21: mol_type = other RNA; note = shRNA21
ggttgaagcagttaattaaag

SEQ ID NO: 22 - RNA, 21 nt, synthetic construct
  source 1..21: mol_type = other RNA; note = shRNA22
cgatattacgcacaactaatg

SEQ ID NO: 23 - RNA, 21 nt, synthetic construct
  source 1..21: mol_type = other RNA; note = shRNA23
agaccatgttgacatactagg

SEQ ID NO: 24 - RNA, 21 nt, synthetic construct
  source 1..21: mol_type = other RNA; note = shRNA24
ggttctttaatctactcaacc

SEQ ID NO: 25 - RNA, 21 nt, synthetic construct
  source 1..21: mol_type = other RNA; note = shRNA25
ggtggtgttgttcagttgact

SEQ ID NO: 26 - RNA, 21 nt, synthetic construct
  source 1..21: mol_type = other RNA; note = shRNA26
gctcgtgttgtacgatcaatt

SEQ ID NO: 27 - RNA, 21 nt, synthetic construct
  source 1..21: mol_type = other RNA; note = shRNA27
agtgtataatgctagtttatt

SEQ ID NO: 28 - RNA, 21 nt, synthetic construct
  source 1..21: mol_type = other RNA; note = shRNA28
ggtacatgtcagcattaaatc

SEQ ID NO: 29 - RNA, 21 nt, synthetic construct
  source 1..21: mol_type = other RNA; note = shRNA29
gttagatgatgatagtcaaca

SEQ ID NO: 30 - RNA, 21 nt, synthetic construct
  source 1..21: mol_type = other RNA; note = shRNA30
gattgtttaggaagtctaatc

SEQ ID NO: 31 - RNA, 21 nt, synthetic construct
  source 1..21: mol_type = other RNA; note = shRNA31
gtctctagtcagtgtgttaat

SEQ ID NO: 32 - RNA, 21 nt, synthetic construct
  source 1..21: mol_type = other RNA; note = shRNA32
ctactaatgttgttattaaag

SEQ ID NO: 33 - RNA, 21 nt, synthetic construct
  source 1..21: mol_type = other RNA; note = shRNA33
ggtgttcttactgagtctaac

SEQ ID NO: 34 - RNA, 21 nt, synthetic construct
  source 1..21: mol_type = other RNA; note = shRNA34
gaaacaaagtgtacgttgaaa

SEQ ID NO: 35 - RNA, 21 nt, synthetic construct
  source 1..21: mol_type = other RNA; note = shRNA35
gttagatttcctaatattaca

SEQ ID NO: 36 - RNA, 21 nt, synthetic construct
  source 1..21: mol_type = other RNA; note = shRNA36
ctgtgatgttgtaataggaat

SEQ ID NO: 37 - RNA, 21 nt, synthetic construct
  source 1..21: mol_type = other RNA; note = shRNA37
ggagtcaaattacattacaca

SEQ ID NO: 38 - RNA, 21 nt, synthetic construct
  source 1..21: mol_type = other RNA; note = shRNA38
gcttactctaataactctatt

SEQ ID NO: 39 - RNA, 21 nt, synthetic construct
  source 1..21: mol_type = other RNA; note = shRNA39
ggtaactgtgatgttgtaata

SEQ ID NO: 40 - RNA, 68 nt, synthetic construct
  source 1..68: mol_type = other RNA; note = synthesis of shRNA1
ttaatacgacctctctgttggattttgacattcaagagatgtcaaatccaacagagaggtcgtattaa

SEQ ID NO: 41 - RNA, 53 nt, synthetic construct
  source 1..53: mol_type = other RNA; note = synthesis of shRNA2
ggttcgcaacttcacacagagtttcaagagaactctgtgtgaagttgcgaacc

SEQ ID NO: 42 - RNA, 53 nt, synthetic construct
  source 1..53: mol_type = other RNA; note = synthesis of shRNA5
ggttcggttgttatatacgatattcaagagatatcgtatataacaaccgaacc

SEQ ID NO: 43 - protein, 740 aa, Homo sapiens
  source 1..740: note = amino acid sequences 1 to 740 of human ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 44 - protein, 23 aa, Homo sapiens
  source 1..23: note = amino acid sequences 741 to 763 of human ACE2
IWLIVFGVVMGVIVVGIVILIFT

SEQ ID NO: 45 - protein, 42 aa, Homo sapiens
  source 1..42: note = amino acid sequences 764 to 805 of human ACE2
GIRDRKKKNKARSGENPYASIDISKGENNPGFQNTDDVQTSF

(19) United States
(12) Patent Application Publication
(10) Pub. No.: US 2023/0203137 A1
(43) Pub. Date: Jun. 29, 2023

(54) PREPARATION METHOD OF ARTIFICIAL ANTIBODY

(71) Applicants: BINGHUAN WENG, HANGZHOU (CN); WOMEN'S HOSPITAL SCHOOL OF MEDICINE ZHEJIANG UNIVERSITY, HANGZHOU (CN); HANGZHOU CHICHUANG BIOTECHNOLOGY CO., LTD., HANGZHOU (CN)

(72) Inventors: Binghuan WENG, Hangzhou (CN); Hefeng HUANG, Hangzhou (CN); Hui WANG, Hangzhou (CN); Lin HE, Hangzhou (CN); Weiping WANG, Hangzhou (CN); Zhiyong ZHU, Hangzhou (CN); Minyue DONG, Hangzhou (CN); Jiali LIN, Hangzhou (CN); Wei XU, Hangzhou (CN); Xufeng YAO, Hangzhou (CN)

(21) Appl. No.: 18/054,212

(22) Filed: Nov. 10, 2022

(30) Foreign Application Priority Data
Nov. 11, 2021 (CN) 202111329857.4
Feb. 22, 2022 (CN) 202210163969.5
May 1, 2022 (CN) 202210491741.9
Aug. 1, 2022 (CN) 202210917136.3

(51) Int. Cl. C07K 16/10; C12N 15/113; C12N 9/48

(52) U.S. Cl. CPC C07K 16/10; C12N 15/1131; C12N 9/485; C12Y 304/17023; C12N 2310/531; C12N 2310/14

(57) ABSTRACT

The present disclosure provides a preparation method of an artificial antibody. The preparation method includes the following steps: screening a target siRNA from a conserved gene or a microsatellite of a coronavirus, synthesizing a small hairpin RNA (shRNA) that has a loop by complementary sense and antisense strands of the siRNA, synthesizing an ACE2 capable of binding to a receptor-binding domain (RBD), and synthesizing the artificial antibody including an shRNA region and an ACE2 region by ligating the ACE2 to sense and antisense strands of the shRNA separately. The bivalent ACE2 is used for neutralization of the RBD and targeted delivery of the shRNA; the shRNA is ligated to the virus through the ACE2 and enters target cells with virus infection, thereby avoiding a side effect of non-specific delivery of the shRNA to uninfected cells, as well as resisting the variant strain and neutralizing the virus with the ACE2.

### CROSS REFERENCE TO RELATED APPLICATION

This patent application claims the benefit and priority of Chinese Patent Application NO: 2021113298574, filed with the China National Intellectual Property Administration on Nov. 11, 2021; NO: 2022101639695, filed with the China National Intellectual Property Administration on Feb. 22, 2022; NO: 2022104917419, filed with the China National Intellectual Property Administration on May 1, 2022; NO: 2022109171363, filed with the China National Intellectual Property Administration on Aug. 1, 2022; the disclosure of which is incorporated by reference herein in its entirety as part of the present applications.

### REFERENCE TO SEQUENCE LISTING

A computer readable XML file entitled “GWP20221000007_sequence listing.xml”, that was created on Nov. 10, 2022, with a file size of about 49,106 bytes, contains the sequence listing for this application, has been filed with this application, and is hereby incorporated by reference in its entirety.

### TECHNICAL FIELD

The present disclosure relates to a preparation method of a broad-spectrum antiviral artificial antibody, and belongs to the field of biopharmaceuticals for prevention and treatment of infectious diseases.

### BACKGROUND

The coronavirus is structurally composed of a single-stranded ribonucleic acid (ssRNA), a spike protein (S protein), a membrane protein (M protein), an envelope protein (E protein), and a nucleocapsid protein (N protein). An N-terminus of the S protein includes an N-terminus domain (S1-NTD) and a receptor-binding domain (S1-RBD).

Human angiotensin-converting enzyme II (ACE2) is a type I transmembrane glycoprotein expressed by target cells. The ACE2 consists of 805 amino acids. Amino acid sequences 1 to 740 are located extracellularly and hence called extracellular ACE2; amino acid sequences 741 to 763 are located in a transmembrane region and hence called transmembrane ACE2; and amino acid sequences 764 to 805 are located intracellularly and hence called intracellular ACE2.

The coronavirus binds to the ACE2 of a target cell through its S1-RBD, undergoes membrane fusion and endocytosis, and enters cells that express the ACE2 or include ACE2 channels. The RBD and the ACE2 act as a ligand and a receptor, respectively, and the ACE2 can neutralize the RBD.

In the prior art, vaccines are mainly designed based on a coronavirus spike protein S1-RBD. After vaccination, the body produces antibodies that neutralize the virus, namely immunoglobulin (Ig). Monomeric Ig includes two heavy chains (H chains) and two light chains (L chains). Monomeric Ig is proteolytically hydrolyzed to generate 1 Fc (containing most of the two H chains) and two Fab (containing a portion of the two H chains and the two L chains). The anti-RBD (Ig-anti-RBD) produced after inoculation with an RBD vaccine of the COVID-19 virus binds to a viral RBD through the two Fab of Ig, thereby preventing the virus from binding to an ACE2 receptor of a target cell through its RBD to infect the target cells.

Small interfering RNA, or siRNA, can regulate gene expression in a manner of participating in RNA interference (RNAi), thus specifically degrading a complementary target messenger RNA (mRNA). A variety of siRNA drugs have been approved and marketed by the Food and Drug Administration (FDA). However, currently the siRNA drug is generally designed using a single strain, the siRNA drugs are prepared using a single-stranded siRNA (antisense RNA), or non-specific delivery of the siRNA drugs is conducted using non-targeted delivery vectors. Moreover, the siRNA generally designed using a single strain may be off-target and ineffective due to the constantly mutating coronavirus. Therefore, it is necessary to compare mutated strains and preferably select a siRNA that is consensus to each strain and does not change with the virus mutation, so that the siRNA can be used for preparing a broad-spectrum siRNA drug for the mutated and mutating strains. More importantly, according to an RNAi mechanism reported by Hre A et al., a double-stranded RNA prepared by mixing the sense RNA and antisense RNA has an efficiency of silencing homologous mRNAs over 100 times higher than that of the single-stranded RNA (antisense RNA). This shows that a correct and effective RNAi technology should prepare drugs using the Short Hairpin RNA (shRNA) containing the sense and antisense siRNAs, rather than the single-stranded siRNA.

In summary, in the present disclosure, an siRNA common to each variant strain is designed, and the siRNA is synthesized into an shRNA duplex; and an ACE2 polypeptide is separately ligated to each end of the shRNA duplex to form a complex of ACE2-shRNA-ACE2, where an shRNA end corresponds to the Fc end of Ig, and two ACE2 ends correspond to the two Fab ends of Ig. The artificial antibody binds to and neutralizes the viral RBD through its two ACE2 the same as Ig binding to and neutralizing the viral RBD through its two Fab, and the shRNA in the artificial antibody further has a broad-spectrum anti-variant strain effect.

### SUMMARY

An objective of the present disclosure is to provide an artificial antibody and a synthesis method and use thereof. A bivalent ACE2 in the synthesized antibody is combined with a viral RBD to form a complex of shRNA-2ACE2-RBD-virus, playing a role of virus neutralization and targeted delivery of an shRNA.

### The Objective of the Present Disclosure is Achieved by the Following Technical Solutions

An shRNA duplex is designed and synthesized, and an ACE2 polypeptide is separately ligated to each end of the shRNA duplex to form a complex of ACE2-shRNA-ACE2, where an shRNA end corresponds to an Fc end of Ig, and two ACE2 ends correspond to two Fab ends of Ig.

The artificial antibody binds to the viral RBD through its ACE2 the same as Fab of Ig binding to the viral RBD, thus blocking a virus from binding to a target cell ACE2 by the viral RBD to infect the target cell, and the shRNA in the artificial antibody further has a broad-spectrum anti-variant strain effect.

According to whole genomes of one coronavirus and its 18 variant strains, an siRNA that does not change with virus mutation is designed, and sense and antisense siRNAs that are complementary as well as a base sequence with a spacer function are synthesized.

Further, the sense and antisense siRNAs are synthesized into an shRNA duplex that has a loop in a middle part formed base separation.

Further, an interference vector is constructed with the shRNA, and mRNA expression, protein expression, and interference effect of the shRNA are detected; after conducting siRNA design, synthesis, screening, iterative design, and verification, the siRNA with a high silencing efficiency is preferably selected.

Further, the preferred siRNA is synthesized into the shRNA, including chemical modifications to increase stability and avoid off-target.

Further, the ACE2 polypeptide is synthesized, including but not limited to a full-length ACE2, a transmembrane ACE2, an intracellular ACE2, an extracellular ACE2, and an amino acid sequence codon-optimized ACE2 polypeptide.

Further, the shRNA is ligated by a coupling method using a disulfide bond, a phosphodiester bond, a phosphorodithioate bond, a thioether bond, an oxime bond, an amide bond, and a maleimide-thiol bond with two ACE2 to synthesize an artificial antibody (ACE2-shRNA-ACE2); alternatively, the ACE2-shRNA-ACE2 is directly synthesized from the amino acid level according to a nucleotide sequence of the shRNA and amino acid sequences of the two ACE2.

Further, the artificial antibody is purified by high-performance liquid chromatography, reversed high-performance liquid chromatography, or ion exchange chromatography.

Further, an antiviral effect of the artificial antibody on two or more different variant strains is detected at the cellular level in vitro, and it is observed whether the artificial antibody has a broad-spectrum anti-variant strain effect targeting the conserved gene; it is tested that whether the artificial antibody has an effect in targeted delivery of the shRNA in animals and whether the artificial antibody can stimulate the host to produce ACE2-Ab.

### The Present Disclosure has the Following Beneficial Effects

According to a mutual binding relationship between a ligand RBD and a receptor ACE2, an artificial antibody (ACE2-shRNA-ACE2) is designed for the first time that the viral RBD is neutralized with the ACE2 and shRNA is delivered by the ACE2. In the artificial antibody, the shRNA end corresponds to the Fc end of Ig, and the two ACE2 ends correspond to the two Fab ends of Ig. The artificial antibody binds to the viral RBD through its ACE2 in a same way that Ig binds to the viral RBD through its Fab, thereby preventing the virus from binding to ACE2 of target cells through its RBD to infect the target cells.

The artificial antibody prepared by ligating the ACE2 polypeptide at two ends of the shRNA duplex includes an shRNA region and a double-stranded ACE2 region. The ACE2 plays a role of delivering the shRNA and then forming a “shRNA-ACE2-RBD-virus” complex after binding to the RBD, such that the shRNA enters the target cells with virus infection. This new method of co-delivering the shRNA with ACE2 and virus avoids a side effect of non-specific delivery of the shRNA to uninfected cells.

Since the siRNA/shRNA is negatively charged and lipid-soluble, with poor permeability and stability; after synthesizing the siRNA/shRNA with the ACE2 into the artificial antibody, the permeability, stability, and easy delivery of the siRNA/shRNA are optimized.

In the ACE2-shRNA-ACE2 synthesized with two ACE2 and one shRNA, since ACE2 binds to RBD bivalently, the virus is essentially neutralized by a bivalent ACE2, which is the same as that Ig neutralizes the virus through two Fab binding to the RBD.

Since the artificial antibody has two ACE2 and two shRNA, and the shRNA can also act as an immunologic adjuvant in addition to anti-virus effects (shRNA is also one of the main adjuvants), the ACE2 in the artificial antibody has strong antigenicity and can stimulate the host to produce high titer of ACE2-Ab. ACE2-Ab can compete with the virus for the ACE2 receptor of the target cell, such that the ACE2-Ab produced by the artificial antibody is capable of preventing virus infection.

In vitro cell experiments show that the artificial antibody is effective against two different variant strains at the same time, indicating an anti-variant strain effect targeting the conserved gene; in vivo animal experiments show that the artificial antibody has an RNAi effect with targeted delivery in animals, and the stimulated ACE2-Ab can inhibit virus infection by blocking the ACE2 receptor.

### BRIEF DESCRIPTION OF THE DRAWINGS

FIG. 1 shows a technical circuit diagram for preparing an artificial antibody in the present disclosure;

FIG. 2 shows a structural representation of the artificial antibody in the present disclosure;

FIG. 3 shows a schematic diagram of the artificial antibody and use thereof in the present disclosure.

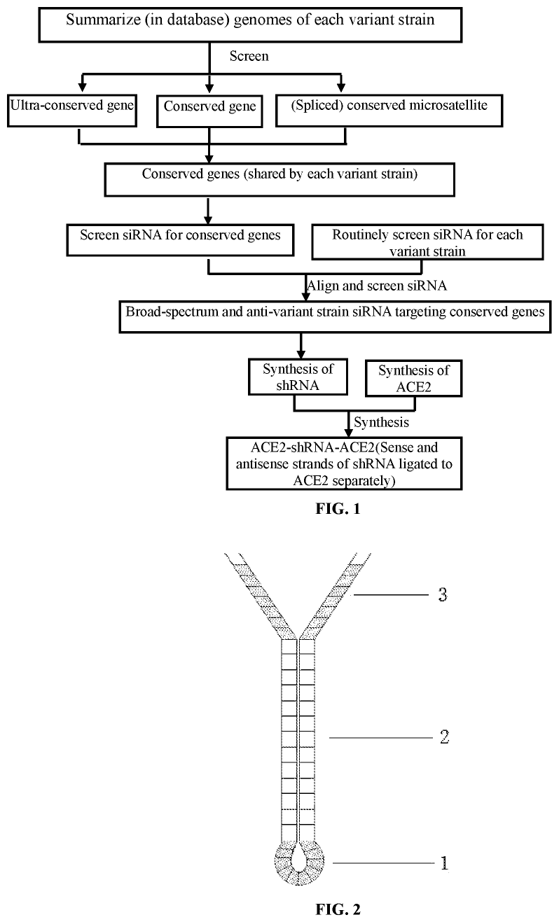

In FIG. 1, after conserved gene screening, broad-spectrum anti-variant strain target siRNA screening by targeting conserved genes, shRNA synthesis, and ACE2 synthesis, the artificial antibody ACE2-shRNA-ACE2 of the present disclosure is synthesized.

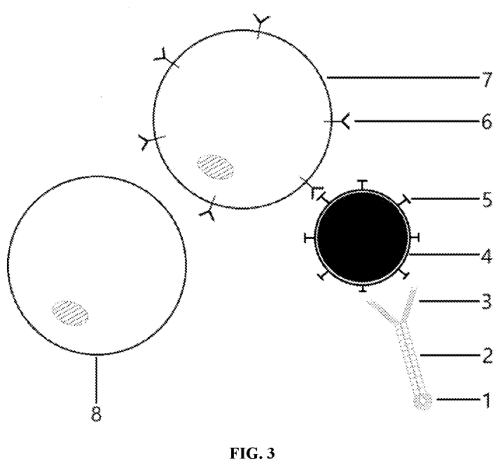

In FIG. 2, 1 is a loop, 2 is an shRNA formed by two complementary sense and antisense strands, and 3 is two ACE2 polypeptides (proteins); the two ACE2 polypeptides are ligated to the sense and antisense strands of the shRNA, respectively. The shRNA is protected by ACE2 and then target-delivered by the ACE2 to the viral RBD or the ACE2 receptor channel, and then specifically enters the target cytoplasm with the viral RBD through the ACE2 channel to degrade the target gene of virus.

In FIG. 3, 1 is the loop formed by the sense and antisense strands of the shRNA through base separation; 2 is the shRNA formed by complementary combination of two sense and antisense strands of siRNA, where the siRNA takes the conserved gene of coronavirus as an interference target and has a targeted gene therapy effect of broad-spectrum anti-variant strain properties; 3 is the ACE2 ligated to the sense and antisense strands of the shRNA; 4 is the coronavirus; 5 is the RBD of a coronavirus S protein; 6 is the cells; 7 is the expressed ACE2 receptors; and 8 is the cells that do not express ACE2. As shown in FIG. 2, the ACE2siRNA is composed of the loop (1), the shRNA (2), and the ACE2 (3); the coronavirus (4) infects cells (7) through specific binding of its RBD (5) to ACE2 receptor (6), but the coronavirus (4) does not infect cells (8) that do not express ACE2; ACE2 (3) in the ACE2siRNA, like ACE2 (6) expressed by cells (7), can also bind to the RBD (5) of coronavirus (4), such that the ACE2 (3) can compete with the ACE2 (6) for binding to the RBD (5), such that the ACE2 (3) can inhibit the virus (4) from infecting the cells (7); the ACE2siRNA plays the role of a vaccine in a later stage, since the ACE2 (3) can stimulate the host to produce anti-ACE2, the anti-ACE2 can block the ACE2 (6), thereby inhibiting the virus (4) from infecting cells (7); more importantly, through bridging of the ACE2 (3), a complex of “shRNA (2)-ACE2 (3)-RBD (5)-virus (4)” is formed, allowing shRNA (2) to enter cell (7) with virus (4) to conduct targeted interference on the replication of virus (4) in cells (7), thereby avoiding toxic and side effects caused by the non-specific entry of the shRNA (2) into cells (8) that are not infected with virus (4).

### DETAILED DESCRIPTION OF THE EMBODIMENTS

Below in conjunction with accompanying drawings 1, 2, and 3, the specific implementation method of the present disclosure is described in detail, but these exemplary descriptions do not constitute any limitation to the protection scope defined by the claims of the present disclosure.

I. Design of siRNA by Targeting Ultra-Conserved Genes, Conserved Genes, or Conserved Microsatellites

1. Design of Ultra-Conserved Genes, Conserved Genes, and Conserved Microsatellites

As shown in FIG. 1 of the technical circuit, a whole genome sequence (cDNA) of β-coronavirus (especially COVID-19 virus and its variant strains) is downloaded from the Genbank database (http://www.NCBI.nlm.nih.gov/genome/), the longest common subsequence is searched in the whole genome sequence to obtain the ultra-conserved genes or conserved genes; with Clustal W software, sequence alignment is conducted on the whole genome downloaded from the Genbank database, the similarity between different sequences is detected, and conserved microsatellite sequences are screened; with MEGA6.0 molecular evolution genetic analysis software, an amino acid germline molecular evolution tree is constructed by using neighbor joining (N-J) on the downloaded coronavirus amino acid sequence, and molecular variation characteristics of the amino acid sequence are analyzed, so as to infer conserved genes sequence.

The following three longest and second longest ultra-conserved subsequences are obtained (identical subsequences without insertion or deletion), with a length of 22 bp to 30 bp, which are comparable to a length of small RNAs, but these three subsequences are not included in higher organisms, especially human beings. A specific sequence is as follows:

SEQ ID NO: 1
(Subsequence 1) = ttaatacgacctctctgttggattttgaca;
(30 bp)
SEQ ID NO: 2
(Subsequence 2) = ggttcgcaacttcacacagagt;
(22 bp)
SEQ ID NO: 3
(Subsequence 3) = caggcgtttgttggttgattaa.
(22 bp)

The following three longest and second longest conserved subsequences are obtained, with a length of 22 bp to 30 bp, which are comparable to a length of small RNAs, but these three conserved subsequences are not included in higher organisms, especially human beings:

SEQ ID NO: 4
(Subsequence 1) = gttttacgacaacgatgttggtttaggaca;
(30 bp)
SEQ ID NO: 5
(Subsequence 2) = ggttcggttgttatatacgata;
(22 bp)
SEQ ID NO: 6
(Subsequence 3) = ggttcagagagtctcctattta.
(22 bp)

The following five conserved microsatellite loci with repeated nucleotides are obtained, where microsatellites are CTCTCT, AGAGAG, AAAAAAA, TATATA, and CACACA, respectively:

2. Screening of siRNA by Targeting Ultra-Conserved Genes, Conserved Genes, or Conserved Microsatellites

With the Clustal W software or other software, gene sequence alignment is conducted on the ultra-conserved genes, conserved genes, and conserved microsatellites with conventionally-screened siRNAs, to detect a similarity between different sequences; multiple pairs of siRNAs are designed, which are the ultra-conserved genes, conserved genes, or conserved microsatellites, as well as RNAi target sites (siRNAs by targeting ultra-conserved genes, conserved genes, or conserved microsatellites are designed).

(1) siRNA by Targeting Ultra-Conserved Genes and Conserved Microsatellites (S1/S2):

SEQ ID NO: 1
(Subsequence 1) = ttaatacgacctctctgttggattttgaca;
(30 bp)
SEQ ID NO: 2
(Subsequence 2) = ggttcgcaacttcacacagagt;
(22 bp)

(2) siRNA by Targeting Conserved Genes and Conserved Microsatellites (S3/S4):

SEQ ID NO: 5
(Subsequence 3) = ggttcggttgttatatacgata;
(22 bp)
SEQ ID NO: 6
(Subsequence 4) = ggttcagagagtctcctattta.
(22 bp)

Through the above design, siRNAs that theoretically resist coronavirus variant strains are obtained with ultra-conserved genes, conserved genes, or conserved microsatellites as interference targets, named siRNA1/2/3/4.

3. Screening of Common Target siRNA of Common Variant Strains

According to a whole genome sequence (cDNA) of β-coronavirus (especially the COVID-19 virus and its variant strains) from the Genbank database (http://www.NCBI.nlm.nih.gov/genome/), with various shRNA online design software (such as http://www.ambion.com/techlib/misc/siRNAtools.html), multiple siRNA candidate sequences are obtained with a length of about 19 nt; based on a Tm value of RNA binding and specificity alignment results, the siRNA is preferably selected. For example, siRNA screening is conducted on E, M, N, ORF1ab, and S genes of 18 variant strains of the COVID-19 virus, including B.1.617.1, B.1.1.529, B.1.1.7, P.1, BA.2, B.1.351, B.1.525, B.1.617.2, C.1.2, B.1.621.1, B.1.621, P.2, N.9, C.37.1, C.37, B.1.427, B.1.351.2, and B.1.351.3, so as to obtain common targets SEQ ID NO: 7 to SEQ ID NO: 39 shown in Table 1; where SEQ ID NO: 7 to SEQ ID NO: 10, SEQ ID NO: 16 to SEQ ID NO: 18, SEQ ID NO: 20 to SEQ ID NO: 22, and SEQ ID NO: 30 to SEQ ID NO: 32 are all preferred siRNAs with a higher score, which can be used as candidate targets for targeting the COVID-19 virus variant strains.

TABLE 1
Common target siRNAs (SEQ ID NO: 7 to SEQ ID NO: 39) screened from 18
COVID-19 virus variant strains

| | | | Common target siRNA | | | |
|---|---|---|---|---|---|---|
| | | | (ss sequence) | | Off- | |
| Variant strain | Region | Position | of variant strains | Score | targets | SEQ ID NO: |
| (1)B.1.617.1, | E gene | 7 | gtggtattcttgctagttaca | 95.1 | 0 | SEQ ID NO: 7 |
| (2)B.1.1.529, | M gene | 35 | ggatttgtcttctacaatttg | 95.7 | 0 | SEQ ID NO: 8 |
| (3)B.1.1.7, | | 146 | cgaacgctttcttattacaaa | 94.8 | 0 | SEQ ID NO: 9 |
| (4)P.1, | | 130 | cactgttgctacatcacgaac | 94.8 | 0 | SEQ ID NO: 10 |
| (5)BA.2, | | 6 | cctagtaataggtttcctatt | 94.7 | 0 | SEQ ID NO: 11 |
| (6)B.1.351, | | 181 | gcgtgtagcaggtgactcagg | 94.3 | 0 | SEQ ID NO: 12 |
| (7)B.1.525, | | 18 | gaccgcttctagaaagtgaac | 93.3 | 0 | SEQ ID NO: 13 |
| (8)B.1.617.2, | | 109 | caaggacctgcctaaagaaat | 91 | 0 | SEQ ID NO: 14 |
| (9)C.1.2, | | 33 | atggatttgtcttctacaatt | 91.0 | 0 | SEQ ID NO: 15 |
| (10)B.1.621.1, | N gene | 137 | cgtagtcgcaacagttcaaga | 100.7 | 0 | SEQ ID NO: 16 |
| (11)B.1.621, | | 12 | ggtggaccctcagattcaact | 99.9 | 0 | SEQ ID NO: 17 |
| (12)P.2, | | 56 | ggttcaccgctctcactcaac | 91.4 | 0 | SEQ ID NO: 18 |
| (13)N.9, | | 39 | cgggaacgtggttgacctaca | 90.4 | 0 | SEQ ID NO: 19 |
| (14)C.37.1, | ORF1ab | 7687 | gcttatgtgtcaacctatact | 106.6 | 0 | SEQ ID NO: 20 |
| (15)C.37, | gene | 8304 | ggttgaagcagttaattaaag | 105.1 | 0 | SEQ ID NO: 21 |
| (16)B.1.427, | | 8613 | cgatattacgcacaactaatg | 104.6 | 0 | SEQ ID NO: 22 |
| (17)B.1.351.2, | | 10519 | agaccatgttgacatactagg | 104.4 | 0 | SEQ ID NO: 23 |
| (18)B.1.351.3 | | 6719 | ggttctttaatctactcaacc | 104.4 | 0 | SEQ ID NO: 24 |
| | | 1805 | ggtggtgttgttcagttgact | 104.3 | 0 | SEQ ID NO: 25 |
| | | 1619 | gctcgtgttgtacgatcaatt | 104.2 | 0 | SEQ ID NO: 26 |
| | | 11328 | agtgtataatgctagtttatt | 103.9 | 0 | SEQ ID NO: 27 |
| | | 4932 | ggtacatgtcagcattaaatc | 103.8 | 0 | SEQ ID NO: 28 |
| | | 2935 | gttagatgatgatagtcaaca | 103.5 | 0 | SEQ ID NO: 29 |
| | S gene | 4 | gattgtttaggaagtctaatc | 103.6 | 0 | SEQ ID NO: 30 |
| | | 29 | gtctctagtcagtgtgttaat | 101.5 | 0 | SEQ ID NO: 31 |
| | | 33 | ctactaatgttgttattaaag | 99.8 | 0 | SEQ ID NO: 32 |
| | | 6 | ggtgttcttactgagtctaac | 99.2 | 0 | SEQ ID NO: 33 |
| | | 68 | gaaacaaagtgtac gttgaaa | 97.1 | 0 | SEQ ID NO: 34 |
| | | 155 | gttagatttcctaatattaca | 96.7 | 0 | SEQ ID NO: 35 |
| | | 7 | ctgtgatgttgtaataggaat | 94.9 | 0 | SEQ ID NO: 36 |
| | | 271 | ggagtcaaattacattacaca | 94.6 | 0 | SEQ ID NO: 37 |
| | | 12 | gcttactctaataactctatt | 94.6 | 0 | SEQ ID NO: 38 |
| | | 2 | ggtaactgtgatgttgtaata | 94.1 | 0 | SEQ ID NO: 39 |

II. Verification of siRNA Function

1. Synthesis of siRNA/shRNA

When siRNA effectively interferes with the mRNA expression of an S gene of SARS-CoV-2, an S protein-deficient virus that loses infectivity is formed. When the siRNA effectively interferes with the mRNA expression of an N gene of the SARS-CoV-2, the packaging and replication of the virus may be inhibited. When the siRNA effectively interferes with the mRNA expression of an ORF1a or ORF1b gene of the SARS-CoV-2, the synthesis of viral RNA polymerase (RdRp) or protein processing enzyme (3CLpro) may be affected. However, the M and E genes are membrane genes of the virus, with an inhibitory effect of their membrane defects on the virus being not obvious. Therefore, siRNA targeting N gene (SEQ ID NO: 16 to SEQ ID NO: 18), siRNA targeting ORF1ab gene (SEQ ID NO: 20 to SEQ ID NO: 22), and siRNA targeting S gene (SEQ ID NO: 30 to SEQ ID NO: 32), and SEQ ID NO: 1 to SEQ ID NO: 2 and SEQ ID NO: 5 to SEQ ID NO: 6 are selected for synthesis. In addition, according to polyclonal restriction enzyme cleavage site of a pSilencer4.1.CMV.neo interference vector, an shRNA template capable of expressing a hairpin structure is designed; each template is composed of two mostly complementary 55 bp single-stranded DNAs, and the single-stranded DNA can be annealed and complementary to form a double-stranded DNA with sticky ends of Bam HI and Hind III restriction sites for ligation with a linearized pSilencer4.1.CMV.neo. According to the designed siRNA and its shRNA template, a company is commissioned to synthesize the siRNA.

2. Construction of shRNA Expression Vector

The shRNA is ligated with the linearized interference vector pSilencer4.1.CMV.neo, and then identified to construct an shRNA expression plasmid, and transformed into DH5a to obtain the shRNA expression vector.

3. Identification of Effect of shRNA Expression (Interference) Vector

According to the synthesized siRNA/shRNA and its constructed expression plasmid, a corresponding target gene is selected for synthesis or PCR amplification, a fluorescent tag vector is constructed, and co-transfected into 293T cells separately with the shRNA expression plasmid, and the cells are identified.

A conventional method of PCR amplification is as follows:

Primer design: upstream and downstream primers are designed, a start codon is added at a 5′-end of the upstream primer, and a homology arm is added to the 5′-end of the primer for homologous recombination with a vector in order to clone an amplification product into pEGFP-N1.

Target gene amplification: gene amplification, product recovery, and purification are conducted according to a gene amplification reaction system and reaction conditions provided by a Shanghai Sangon kit to obtain the amplification product.

Linearization of pEGFP-N1: a DH5a strain containing a pEGFP-N1 plasmid is resuscitated, the plasmid is extracted by the kit, the concentration is determined, restriction digestion is conducted, and a linearized vector is identified and recovered by 0.8% agarose gel electrophoresis.

An amplified target gene is ligated to a fluorescent tag vector (pEGFP-N1): the ligation is conducted with a GenScript's homologous recombination kit, and a ligated product can be stored at−20° C. for later use or transformed immediately.

Identification of effects of an shRNA interference vector: the interference vector (pSilencer-shRNA) and the fluorescent tag vector (pEGFP-N/S/ORF1ab) are co-transfected into 293T cells, where the interference vector and the tag vector are in a mass ratio of 1:2, while a control is set up; the fusion expression of a GFP protein in the cells is observed 48 h after the transfection, and an interference effect is evaluated according to a fluorescence intensity.

Flow cytometry: to quantitatively analyze the interference effects of different interference vectors, flow cytometry is conducted to analyze a proportion of fluorescent protein-expressing cells in the total number of cells.

Western bolt analysis: (1) cell collection and lysis: cells are lysed with RIPA; (2) SDS-PAGE protein electrophoresis: an SDS-PAGE gel is prepared, a sample is added to an equal volume of a 2×SDS buffer, boiled in boiling water for 5 min, treated in an ice bath for 2 min, and then centrifuged at 12,000×g for 10 min; (3) Western blot detection: after conducting transferring, blocking, primary antibody binding, washing, secondary antibody binding, and color development, results are observed.

RT-PCR detection of mRNA: relative fluorescence quantitative RT-PCR is conducted to detect a relative expression level of the target genes in transfected cells; according to a standard curve, a copy number of the target gene and a B-actin reference gene is converted from a CT value; a relative expression level of the viral gene mRNA (the number of copies of the target gene/the number of copies of the B-actin) is corrected with the B-actin reference gene, such that an interference effect is quantitatively evaluated.

4. Obtaining siRNA/shRNA with a High Silencing Efficiency

After design, synthesis, screening, iterative design, resynthesis, and verification at the cellular level, siRNAs/shRNAs with a higher silencing efficiency are preferably obtained. The siRNAs/shRNAs have sequences of SEQ ID NO: 1 (named shRNA1, the same below), SEQ ID NO: 2 (shRNA2), SEQ ID NO: 5 (shRNA5), SEQ ID NO: 16 (shRNA16) targeting the N gene, SEQ ID NO: 21 (shRNA21) targeting the ORF1ab gene, and SEQ ID NO: 30 (shRNA30) targeting the S gene, with silencing efficiencies of 78%, 76%, 88%, 89%, 84%, and 90%, respectively.

III. Synthesis of shRNA Targeting Conserved Genes

According to the common targets (SEQ ID NO: 1 to SEQ ID NO: 39), and the preferred sequences with a high silencing efficiency, SEQ ID NO: 5 (shRNA5), SEQ ID NO: 16 (shRNA16), SEQ ID NO: 21 (shRNA21), and SEQ ID NO: 30 (shRNA30), the sequences with a high silencing efficiency are synthesized by a biological company. Each shRNA synthesizes 2 complementary oligonucleotide polypeptide siRNAs of 19 nt to 25 nt, and synthesizes a 9 nt base sequence that acts as a spacer; the two siRNAs and the base sequence are ligated into a shRNAduplex that has a loop in a middle part formed base separation; each single strand of the shRNA duplex can be ligated to an ACE2.

For example, SEQ ID NO: 1 (shRNA1), SEQ ID NO: 2 (shRNA2), and SEQ ID NO: 5 (shRNA5) are synthesized into 5′-ttaatacgacctctctgttggattttgacattcaagagatgtcaaatccaacagagaggtcgtattaa-3′ (SEQ ID NO: 40), 5′-ggttcgcaacttcacacagagtttcaagagaactctgtgtgaagttgcgaacc-3′ (SEQ ID NO: 41) and 5′-ggtt cggttgttatatacgatattcaagagatatcgtatataacaaccg aacc-3′ (SEQ ID NO: 42), where “TTCAAGAGA” is a loop, and left and right sides thereof are complementary sense and antisense strands, and then SEQ ID NO: 40 to SEQ ID NO: 42 show the synthesis of shRNA1, shRNA2, and shRNA5, respectively. Similarly, preferably siRNAs with a high silencing efficiency are synthesized into shRNAs, and ACE2 or a polypeptide thereof is ligated at a 3′-end and/or a 5′-end of the shRNA separately.

IV. Design and Synthesis of Targeted Delivery Vector ACE2

1. Amino Acid Sequences of ACE2

The human ACE2 gene sequence information was searched from the GeneBank database (http://www.ncbi.nlm.gov/genbank); the ACE2 consists of 805 amino acids, where amino acid sequences 1 to 740 (SEQ ID NO: 43) are located extracellularly, amino acid sequences 741 to 763 (SEQ ID NO: 44) are located in a transmembrane region, and amino acid sequences 764 to 805 (SEQ ID NO: 45) are located intracellularly. Among the 20 amino acids that make up the ACE2, leucine accounts for 9.4%, cysteine and histidine account for 1.0% and 2.0%, respectively, and negatively-charged amino acid residues (aspartate+glutamate) and positively-charged amino acid residues (arginine+lysine) as a balance. SARS-CoV and SARS-CoV-2 interact with an extracellular catalytic domain of the ACE2 through viral RBD. This interaction can lead to endocytosis and membrane fusion, such that the SARS-CoV enters cells expressing the ACE2 or containing ACE2 channels.

2. Receptor Function of ACE2

ACE2 is a lipid-soluble type I transmembrane glycoprotein with an amino-terminal catalytic domain and a carboxyl-terminal domain. The N-terminus is outside the cell membrane and the C-terminus is inside the cell membrane. This glycoprotein is divided into an N-terminal signal peptide region, a carboxypolypeptidase activation region, and a transmembrane region. When the Spike protein of coronavirus contacts a tip of a subdomain I of the catalytic domain of ACE2 (without affecting a subdomain II and a peptidase activity), an outer domain of the ACE2 is cleaved and the transmembrane domain is internalized, enabling further virus-host cell fusion. Therefore, it is believed that the transmembrane region is involved in the transport of virus-receptor complexes from the cell membrane to the cytoplasm.

3. Design of ACE2 of the Present Disclosure

From an amino acid sequence and a receptor function of the ACE2, it can be seen that there are ACE2 receptor channels in the cell wall and cell membrane of ACE2-expressing cells. Full-length ACE2, transmembrane region ACE2, intracellular ACE2, and extracellular ACE2 each are a membrane-penetrating polypeptide with a function of targeted delivery of siRNA. The N-terminus of extracellular ACE2 has a function of neutralizing virus by binding to the coronaviral RBD. Therefore, a full-length ACE2, a transmembrane ACE2 with amino acid sequences 741 to 763, an intracellular ACE2 with amino acid sequences 764 to 805, an extracellular ACE2 with amino acid sequences 1 to 740, and an ACE2 polypeptide with optimized amino acid sequences can be designed and synthesized as a targeted delivery vector for siRNA.

4. Synthesis of ACE2 of the Present Disclosure

Two amino acids are dehydrated and condensed to form peptide bonds, and multiple amino acid residues are ligated by the peptide bonds to form polypeptides. A company can be entrusted to automatically synthesize peptides by a peptide synthesizer. Basically, amino acids are added in order according to a sequence of the polypeptide to be synthesized, such that the peptide chain is gradually extended from the C-terminal to the N-terminal residues; each amino acid residue is required to be condensed in the form of protection at one end and activation at the other end, and temporary protection groups on the amino group are removed after each round of peptide chain elongation, until all amino acid sequences of the target polypeptide are condensed. At present, a commonly used reaction for solid-phase synthesis of the polypeptides includes: in a closed explosion-proof glass reactor, amino acids are continuously added from the C-terminus-carboxy terminus to the N-terminus-amino terminus according to a known sequence, and synthesis is conducted to finally obtain the polypeptides. A synthesis method includes: (1) deprotection: removing the protective group of the amino group with an alkaline solvent; (2) activation and cross-linking: activating a carboxyl group of a next amino acid, cross-linking an activated single carboxyl group with a free amino group to form a peptide bond; and repeating these two steps until the polypeptide is synthesized.

V. Synthesis of Artificial Antibody with ACE2 and shRNA

1. Design of Artificial Antibody (2ACE2-shRNA)

Take the synthesis of amino acid sequence-optimized extracellular ACE2 (SEQ ID NO: 43) polypeptide, and shRNA5 (SEQ ID NO: 5), shRNA16 (SEQ ID NO: 16), shRNA21 (SEQ ID NO: 21), or shRNA30 (SEQ ID NO: 30) as an example: one end of the sense and antisense strands of the synthesized shRNA is ligated to the loop (5′-TTCAAGAGA-3′), and the other end is ligated to ACE2 polypeptide (C-terminus), so as to obtain a structure of “extracellular ACE2-siRNA sense strand-loop-siRNA antisense strand-extracellular ACE2”. The complementary sense and antisense siRNAs can form a shRNA duplex. As shown in FIG. 2, it is a hairpin ligation product with two ACE2 polypeptides, siRNA sense and antisense strands, and a loop, which is the ACE2-shRNA-ACE2, abbreviated as 2ACE2-shRNA. In a same way, two ACE2 (SEQ ID NO: 44 and SEQ ID NO: 45) are ligated with the shRNA to form ACE2-shRNA-ACE2.

The products synthesized from the above sequences are expressed as 2ACE2-shRNA5, 2ACE2-shRNA16, 2ACE2-shRNA21, and 2ACE2-shRNA30, respectively. Since the viral RBD infects cells through the C-terminus binding to the N-terminus of ACE2, and the binding of ACE2 polypeptide to siRNA can increase the permeability, stability and interference effect of siRNA. Accordingly, as shown in FIG. 3, ACE2 in this design can neutralize the virus and prevent virus infection, and can conduct targeted delivery on siRNA/shRNA to the viral RBD to form a complex of “shRNA-ACE2-RBD-virus”. As a result, the shRNA is delivered by the virus, and the shRNA enters the target cells with virus infection, playing a role of targeted interference.

2. Synthesis of Extracellular ACE2-shRNA-Extracellular ACE2 (ACE2-shRNA)

An artificial antibody is synthesized according to the design (FIG. 2), by a conventional synthesis method of polypeptide and oligonucleotide, the polypeptide (ACE2) and oligonucleotide (shRNA/siRNA) are coupled to form a conjugate with a carboxyhydrazone bond, a disulfide bond, a phosphodiester bond, a phosphorodithioate bond, a thioether bond, an oxime bond, an amide bond, and a maleimide-thiol bond. The sense strand (5′-end and 3′-end) or antisense strand (3′-end) of polypeptides and oligonucleotides can be non-covalently or covalently cross-linked with a firmer covalent bond, a looser ionic bond, a hydrophobic bond, or the carboxyhydrazone bonds with a spacer arm to synthesize a polypeptide-oligonucleotide conjugate (POCs). At present, the POCs are generally synthesized by covalent crosslinking-liquid phase fragment synthesis, and various POCs are prepared. The method includes the following steps: synthesizing a polypeptide and an oligonucleotide separately on a solid-phase substrate, simultaneously peeling the polypeptide and the oligonucleotide from the solid-phase substrate, and coupling peeled polypeptide and oligonucleotide in a solution by a reactive group. Synthesis of POCs mainly includes: (1) Maleimide-thiol bond coupling: maleimide is modified on the polypeptide or oligonucleotide, thiol is modified on another monomer, and the two monomers are added into a same solution to obtain the POCs after a reaction. (2) Disulfide bond or thioether bond coupling: 5′- or 3′-positions of the oligonucleotide is modified with a thiol group, and then reacted with a polypeptide whose C-terminus is modified with a bromoacetyl group in a buffer solution of pH 7.0; the disulfide bond coupling can be directly oxidized by two thiol groups, or the thiol group can be activated by an activator such as dipyridyl disulfide and then coupled with another oligomer containing a thiol group; the disulfide bonds are commonly used to synthesize a conjugate of siRNA and polypeptides. (3) Oxime bond coupling: the aldehyde group reacts with the amino group to produce oxime; the reaction conditions are mild, with a high reaction efficiency, and a coupling product of double-stranded DNA and a specific polypeptide can be directly generated; meanwhile, two polypeptides can be simultaneously ligated to the 5′- and 3′-end of the nucleic acid through an oxime bond, by a bifunctional oligonucleotide with a polypeptide or a carbohydrate. This method does not require various protection processes and can be completed in one step, which is used to synthesize a “peptide-oligonucleotide-peptide” product. Specifically, the aldehyde group is introduced into the 5′- and 3′-end of the oligonucleotide, and then reacted with a hydroxylamine-modified polypeptide to obtain a “peptide-oligonucleotide-peptide” with a high yield. This one-step reaction of bifunctionalized oligonucleotides with polypeptides does not require any protection strategies and cross-linking reagents, and has a high yield under the slightly acidic environment. (4) Amide bond coupling: an oligomer containing activated carboxylic acid or thioester is reacted with another polymer modified with an amino group to obtain a product. (5) Hydrazone bond coupling: a hydrazine group is introduced into the polypeptide, a citric acid buffer with a pH value of 3 to 5 is added, and the mixture is reacted with an oligonucleotide modified with an acetaldehyde group. Thus, POCs ligated by hydrazone bonds are obtained.

3. Purification of Artificial Antibody (ACE2-shRNA)

Chromatographic methods are most commonly used for purification and analysis of the conjugate of polypeptides and oligonucleotides. According to complexity of the conjugates, different chromatographic methods should be selected for separation. The main methods include high-performance liquid chromatography (HPLC), reverse high-performance liquid chromatography (RP-HPLC), ion exchange chromatography (IEC, generally anion exchange chromatography), or two or more of which are used in series, which is conducted according to operating instructions.

4. Synthesis Method of Other Compounds for Delivery of shRNA by Targeting ACE2

Similarly, shRNA can be ligated with extracellular ACE2, transmembrane ACE2, intracellular ACE2, or codon-optimized ACE2 polypeptide to form compounds, including but not limited to “transmembrane ACE2-shRNA-transmembrane ACE2”, and “intracellular ACE2-shRNA-intracellular ACE2”; alternatively, the shRNA/siRNA is inserted into the middle of ACE2 polypeptide to synthesize “transmembrane ACE2-shRNA-extracellular ACE2”, and “intracellular ACE2-shRNA-extracellular ACE2”; similarly, compounds with ACE2 or ACE2-optimized polypeptides as targeted delivery vectors are designed, including but not limited to “ACE2-siRNA, extracellular ACE2-siRNA, transmembrane ACE2-siRNA, and intracellular ACE2-siRNA”.

VI. Validation of Artificial Antibody (ACE2-shRNA)

1. In Vitro Verification of Broad-Spectrum Antiviral Effect

(1) Preparation of Virus Solution

The virus strains were added to a DMEM medium (10% FBS) of Vero E6 cells grown to 30% confluence, and incubated in a 36° C., 5% CO₂ incubator for 5 d to 7 d; when an cytopathic effect (CPE) occurred, the virus was isolated and then prepared by a medium into a 10³ TCID₅₀/ml to 10⁵ TCID₅₀/ml virus solution for later use. According to this, virus solutions of two variant strains B.1.617.1 and B.1.617.2 of the COVID-19 virus were prepared separately to verify whether the ACE2-shRNA was effective against two or more variant viruses containing a same conserved gene, so as to prove whether the shRNA/siRNA of the present disclosure had a broad-spectrum antiviral effect.

(2) Co-Culture of Artificial Antibody (ACE2-shRNA) with Virus

An experimental group and a control group were set up to test an effect of the compounds against the B.1.617.1 and B.1.617.2. Each group was inoculated with a 8-well plate, and 2×10⁵ Vero-E6 cells and 2 mL of a DMEM medium (10% FBS) were added to each well, and then incubated in a 36° C. and 5% CO₂ incubator to 30% confluence, followed by changing the medium; meanwhile, the tested compound and the virus solutions of B.1.617.1, and B.1.617.2 strains were added.

The experimental group included: (1) a 2ACE2-shRNA5 group (0.1 nmol 2ACE2-shRNA+0.6 ml virus solution); the control groups included: a naked shRNA5 group (0.1 nmol naked shRNA5+0.6 ml virus solution), a positive control group (0.6 ml virus solution), and a negative control group (0.6 ml DMEM culture solution) (Tables 1 to 6). (2) A 2ACE2-shRNA16 group, a 2ACE2-shRNA21 group (0.1 nmol 2ACE2-shRNA16/21+0.6 ml virus solution), and an ACE2 group (0.1 nmol ACE2+0.6 ml virus solution); the control group is the same as that in (1). The results were shown in Tables 1a to 6a. After 1 h, 24 h, and 72 h of incubation, a supernatant was collected from each group, and then diluted at 1:4, 1:12, 1:36, 1:108, 1:324, 1:972, 1:2916, and 1:8748 to conduct RT-PCR detection.

(3) Real-Time Fluorescent RT-PCR Detection of Viral RNA in Each Group

Viral nucleic acid extraction and nucleic acid (ORF1ab/N) detection were conducted according to kit instructions.

(4) Detection Results of Viral RNA

1) Test Results of Strain B.1.617.1

As shown in Table 1, after each group of cells was cultured for 1 h, the 2ACE2-shRNA5 group, naked shRNA5 group, positive control group, and negative control group had viral RNA detection results of 1:12, 1:12, and 1:108, and negative, respectively.

As shown in Table 2, after each group of cells was cultured for 24 h, the 2ACE2-shRNA5 group, naked shRNA5 group, positive control group, and negative control group had viral RNA detection results of 1:36, 1:108, 1:324, and negative, respectively. There was a significant difference between the 2ACE2-shRN5A group and the positive control group (p<0.05).

As shown in Table 3, after each group of cells was cultured for 72 h, the 2ACE2-shRNA5 group, naked shRNA5 group, positive control group, and negative control group had viral RNA detection results of 1:324, 1:8748, 1:8748, and negative, respectively. There was a significant difference between the 2ACE2-shRNA5 group and the positive control group (p<0.01).

Tables 1 to 3 showed that the 2ACE2-shRNA5 group had an obvious anti-B.1.617.1 effect, indicating that shRNA ligated to ACE2 could be delivered to target cells for RNA interference. However, the shRNA that was not ligated to ACE2 could not enter the target cells, and could not play a role of RNA interference extracellularly.

Tables 1a to 3a showed detection results of viral RNA in each medium of the 2ACE2-shRNA16 group and the 2ACE2-shRNA21 group, which were significantly lower than those of the positive control group, while ACE2 was favorable for virus growth.

TABLE 1
Virus RNA RT-PCR detection results (+/−) in medium after
2ACE2-shRNA5 co-cultured with strain B.1.617.1 for 1 h

| | Viral RNA detection results of different | | | | | | | |
|---|---|---|---|---|---|---|---|---|
| | dilutions in medium (+/−) | | | | | | | |
| Group | 1:4 | 1:12 | 1:36 | 1:108 | 1:324 | 1:972 | 1:2916 | 1:8748 |
| 2ACE2- | + | + | − | − | − | − | − | − |
| shRNA5 | | | | | | | | |
| naked | + | + | − | − | − | − | − | − |
| shRNA5 | | | | | | | | |
| Positive | + | + | + | + | − | − | − | − |
| control | | | | | | | | |
| Negative | − | − | − | − | − | − | − | − |
| control | | | | | | | | |

TABLE 1a
Virus RNA RT-PCR detection results (+/−) in medium after
2ACE2-shRNA16/21 co-cultured with strain B.1.617.1 for 1 h

| | Viral RNA detection results of different | | | | | | | |
|---|---|---|---|---|---|---|---|---|
| | dilutions in medium (+/−) | | | | | | | |
| Group | 1:4 | 1:12 | 1:36 | 1:108 | 1:324 | 1:972 | 1:2916 | 1:8748 |
| 2ACE2- | + | − | − | − | − | − | − | − |
| shRNA16 | | | | | | | | |
| 2ACE2- | + | − | − | − | − | − | − | − |
| shRNA21 | | | | | | | | |
| naked | + | − | − | − | − | − | − | − |
| shRNA16 | | | | | | | | |
| naked | + | − | − | − | − | − | − | − |
| shRNA21 | | | | | | | | |
| ACE2 | + | + | − | − | − | − | − | − |
| Positive | + | + | − | − | − | − | − | − |
| control | | | | | | | | |
| Negative | − | − | − | − | − | − | − | − |
| control | | | | | | | | |

TABLE 2
Virus RNA RT-PCR detection results (+/−) in medium after
2ACE2-shRNA5 co-cultured with strain B.1.617.1 for 24 h

| | Viral RNA detection results of different | | | | | | | |
|---|---|---|---|---|---|---|---|---|
| | dilutions in medium (+/−) | | | | | | | |
| Group | 1:4 | 1:12 | 1:36 | 1:108 | 1:324 | 1:972 | 1:2916 | 1:8748 |
| 2ACE2- | + | + | + | − | − | − | − | − |
| shRNA5 | | | | | | | | |
| 2naked | + | + | + | + | − | − | − | − |
| shRNA5 | | | | | | | | |
| Positive | + | + | + | + | + | − | − | − |
| control | | | | | | | | |
| Negative | − | − | − | − | − | − | − | − |
| control | | | | | | | | |

TABLE 2a
Virus RNA RT-PCR detection results (+/−) in medium after
2ACE2-shRNA16/21 co-cultured with strain B.1.617.1 for 24 h

| | Viral RNA detection results of different | | | | | | | |
|---|---|---|---|---|---|---|---|---|
| | dilutions in medium (+/−) | | | | | | | |
| Group | 1:4 | 1:12 | 1:36 | 1:108 | 1:324 | 1:972 | 1:2916 | 1:8748 |
| 2ACE2- | + | + | − | − | − | − | − | − |
| shRNA16 | | | | | | | | |
| 2ACE2- | + | + | − | − | − | − | − | − |
| shRNA21 | | | | | | | | |
| naked | + | + | + | + | − | − | − | − |
| shRNA16 | | | | | | | | |
| naked | + | + | + | + | − | − | − | − |
| shRNA21 | | | | | | | | |
| ACE2 | + | + | + | + | + | + | − | − |
| Positive | + | + | + | + | + | − | − | − |
| control | | | | | | | | |
| Negative | − | − | − | − | − | − | − | − |
| control | | | | | | | | |

TABLE 3
Virus RNA RT-PCR detection results (+/−) in medium after
2ACE2-shRNA5 co-cultured with strain B.1.617.1 for 72 h

| | Viral RNA detection results of different | | | | | | | |
|---|---|---|---|---|---|---|---|---|
| | dilutions in medium (+/−) | | | | | | | |
| Group | 1:4 | 1:12 | 1:36 | 1:108 | 1:324 | 1:972 | 1:2916 | 1:8748 |
| 2ACE2- | + | + | + | + | + | − | − | − |
| shRNA5 | | | | | | | | |
| 2naked | + | + | + | + | + | + | + | + |
| shRNA5 | | | | | | | | |
| Positive | + | + | + | + | + | + | + | + |
| control | | | | | | | | |
| Negative | − | − | − | − | − | − | − | − |
| control | | | | | | | | |

TABLE 3a
Virus RNA RT-PCR detection results (+/−) in medium after
2ACE2-shRNA16/21 co-cultured with strain B.1.617.1 for 72 h

| | Viral RNA detection results of different | | | | | | | |
|---|---|---|---|---|---|---|---|---|
| | dilutions in medium (+/−) | | | | | | | |
| Group | 1:4 | 1:12 | 1:36 | 1:108 | 1:324 | 1:972 | 1:2916 | 1:8748 |
| 2ACE2- | + | + | + | − | − | − | − | − |
| shRNA16 | | | | | | | | |
| 2ACE2- | + | + | + | − | − | − | − | − |
| shRNA21 | | | | | | | | |
| naked | + | + | + | + | + | + | − | − |
| shRNA16 | | | | | | | | |
| naked | + | + | + | + | + | + | − | − |
| shRNA21 | | | | | | | | |
| ACE2 | + | + | + | + | + | + | + | + |
| Positive | + | + | + | + | + | + | + | − |
| control | | | | | | | | |
| Negative | − | − | − | − | − | − | − | − |
| control | | | | | | | | |

2) Test Results of Strain B.1.617.2

As shown in Table 4, after each group of cells was cultured for 1 h, the 2ACE2-shRNA5 group, naked shRNA5 group, positive control group, and negative control group had viral RNA detection results of 1:12, 1:36, 1:36, and negative, respectively. There was a significant difference between the 2ACE2-shRNA5 group and the positive control group (p<0.05).

As shown in Table 5, after each group of cells was cultured for 24 h, the 2ACE2-shRNA5 group, naked shRNA5 group, positive control group, and negative control group had viral RNA detection results of 1:36, 1:108, 1:324, and negative, respectively. There was a significant difference between the ACE2-shRNA group and the positive control group (p<0.01).

As shown in Table 6, after each group of cells was cultured for 72 h, the 2ACE2-shRNA5 group, naked shRNA5 group, positive control group, and negative control group had viral RNA detection results of 1:108, 1:8748, 1:8748, and negative, respectively. There was a significant difference between the 2ACE2-shRNA5 group and the positive control group (p<0.01).

Tables 4 to 6 showed that the 2ACE2-shRNA5 group had an obvious anti-B.1.617.2 effect, indicating that shRNA or siRNA ligated to ACE2 could be delivered to target cells for RNA interference. However, the shRNA that was not ligated to RBD could not enter the target cells, and could not play a role of RNA interference.

The results in Tables 4a to 6a were clearly consistent with the results in Tables 4 to 6, the detection results of viral RNA in each medium of the 2ACE2-shRNA16 group and the 2ACE2-shRNA21 group were significantly lower than those of the positive control group, while ACE2 was favorable for virus growth.

Tables 1 to 6 and 1a to 6a showed that the 2ACE2-shRNA5, 2ACE2-shRNA16, and 2ACE2-shRNA21 had anti-B.1.617.1 and B.1.617.2 effects, indicating a broad-spectrum anti-variant strain effect.

TABLE 4
Virus RNA RT-PCR detection results (+/−) in medium after
2ACE2-shRNA5 co-cultured with strain B.1.617.2 for 1 h

| | Viral RNA detection results of different | | | | | | | |
|---|---|---|---|---|---|---|---|---|
| | dilutions in medium (+/−) | | | | | | | |
| Group | 1:4 | 1:12 | 1:36 | 1:108 | 1:324 | 1:972 | 1:2916 | 1:8748 |
| 2ACE2- | + | + | − | − | − | − | − | − |
| shRNA5 | | | | | | | | |
| naked | + | + | + | − | − | − | − | − |
| shRNA5 | | | | | | | | |
| Positive | + | + | + | − | − | − | − | − |
| control | | | | | | | | |
| Negative | − | − | − | − | − | − | − | − |
| control | | | | | | | | |

TABLE 4a
Virus RNA RT-PCR detection results (+/−) in medium after
2ACE2-shRNA16/21 co-cultured with strain B.1.617.2 for 1 h

| | Viral RNA detection results of different | | | | | | | |
|---|---|---|---|---|---|---|---|---|
| | dilutions in medium (+/−) | | | | | | | |
| Group | 1:4 | 1:12 | 1:36 | 1:108 | 1:324 | 1:972 | 1:2916 | 1:8748 |
| 2ACE2- | + | − | − | − | − | − | − | − |
| shRNA16 | | | | | | | | |
| 2ACE2- | + | − | − | − | − | − | − | − |
| shRNA21 | | | | | | | | |
| naked | + | − | − | − | − | − | − | − |
| shRNA16 | | | | | | | | |
| naked | + | − | − | − | − | − | − | − |
| shRNA21 | | | | | | | | |
| ACE2 | + | + | + | − | − | − | − | − |
| Positive | + | + | − | − | − | − | − | − |
| control | | | | | | | | |
| Negative | − | − | − | − | − | − | − | − |
| control | | | | | | | | |

TABLE 5
Virus RNA RT-PCR detection results (+/−) in medium after
2ACE2-shRNA5 co-cultured with strain B.1.617.2 for 24 h

| | Viral RNA detection results of different | | | | | | | |
|---|---|---|---|---|---|---|---|---|
| | dilutions in medium (+/−) | | | | | | | |
| Group | 1:4 | 1:12 | 1:36 | 1:108 | 1:324 | 1:972 | 1:2916 | 1:8748 |
| 2ACE2- | + | + | + | − | − | − | − | − |
| shRNA5 | | | | | | | | |
| naked | + | + | + | + | + | − | − | − |
| shRNA5 | | | | | | | | |
| Positive | + | + | + | + | + | − | − | − |
| control | | | | | | | | |
| Negative | − | − | − | − | − | − | − | − |
| control | | | | | | | | |

TABLE 5a
Virus RNA RT-PCR detection results (+/−) in medium after
2ACE2-shRNA16/21 co-cultured with strain B.1.617.2 for 24 h

| | Viral RNA detection results of different | | | | | | | |
|---|---|---|---|---|---|---|---|---|
| | dilutions in medium (+/−) | | | | | | | |
| Group | 1:4 | 1:12 | 1:36 | 1:108 | 1:324 | 1:972 | 1:2916 | 1:8748 |
| 2ACE2- | + | + | + | − | − | − | − | − |
| shRNA16 | | | | | | | | |
| 2ACE2- | + | + | − | − | − | − | − | − |
| shRNA21 | | | | | | | | |
| naked | + | + | + | + | + | − | − | − |
| shRNA16 | | | | | | | | |
| naked | + | + | + | + | − | − | − | − |
| shRNA21 | | | | | | | | |
| ACE2 | + | + | + | + | + | + | + | − |
| Positive | + | + | + | + | + | + | − | − |
| control | | | | | | | | |
| Negative | − | − | − | − | − | − | − | − |
| control | | | | | | | | |

TABLE 6
Virus RNA RT-PCR detection results (+/−) in medium after
2ACE2-shRNA5 co-cultured with strain B.1.617.2 for 72 h

| | Viral RNA detection results of different | | | | | | | |
|---|---|---|---|---|---|---|---|---|
| | dilutions in medium (+/−) | | | | | | | |
| Group | 1:4 | 1:12 | 1:36 | 1:108 | 1:324 | 1:972 | 1:2916 | 1:8748 |
| 2ACE2- | + | + | + | + | − | − | − | − |
| shRNA5 | | | | | | | | |
| naked | + | + | + | + | + | + | + | + |
| shRNA5 | | | | | | | | |
| Positive | + | + | + | + | + | + | + | + |
| control | | | | | | | | |
| Negative | − | − | − | − | − | − | − | − |
| control | | | | | | | | |

TABLE 6a
Virus RNA RT-PCR detection results (+/−) in medium after
2ACE2-shRNA16/21 co-cultured with strain B.1.617.2 for 72 h

| | Viral RNA detection results of different | | | | | | | |
|---|---|---|---|---|---|---|---|---|
| | dilutions in medium (+/−) | | | | | | | |
| Group | 1:4 | 1:12 | 1:36 | 1:108 | 1:324 | 1:972 | 1:2916 | 1:8748 |
| 2ACE2- | + | + | + | + | − | − | − | − |
| shRNA16 | | | | | | | | |
| 2ACE2- | + | + | + | − | − | − | − | − |
| shRNA21 | | | | | | | | |
| naked | + | + | + | + | + | + | + | − |
| shRNA16 | | | | | | | | |
| naked | + | + | + | + | + | + | + | − |
| shRNA21 | | | | | | | | |
| ACE2 | + | + | + | + | + | + | + | + |
| Positive | + | + | + | + | + | + | + | − |
| control | | | | | | | | |
| Negative | − | − | − | − | − | − | − | − |
| control | | | | | | | | |

2. In Vivo Verification of Targeted Delivery Function of ACE2

(1) Animal Grouping and Inoculation

Animal grouping: SPF-grade female BALB/c mice aged 6 to 8 weeks and weighed about 40 g were randomly divided into a 2ACE2-shRNA5 group (inoculated with 2ACE2-shRNA5+B.1.617.2), an shRNA5 group (inoculated with shRNA5+B.1.617.2), a positive control group (inoculated with B.1.617.2+physiological saline), and a negative control group (inoculated with physiological saline only), with 20 mice in each group.

Animal inoculation: the mice each were inoculated with 40 μl of a B.1.617.2 strain virus solution with a titer of 10⁵/ml TCID₅₀ by nasal spray, while the negative control group was inoculated with 40 μl of a normal saline by nasal spray. The mice were anesthetized by intraperitoneal injection of a 5% chloral hydrate solution, and 0.1 nmol of the ACE2-shRNA and the shRNA were slowly injected into the trachea of the mice separately. On the 7th day after infection, 10 mice in each group were sacrificed for virus detection; the remaining 10 mice in each group were used to observe antibodies.

(2) Detection of Virus at Percentage of Median Tissue Culture Infective Dose (TCID₅₀) of Cells

A 10% homogenate was prepared from a lung tissue of the mice sacrificed on the 7th day after infection, and 100 μl of the homogenate was centrifuged to remove a supernatant, the homogenate was diluted 10-fold successively, and inoculated in a 96-well plate with VeroE6 growing in a single layer at 30 μl per well and 4 wells per dilution; the homogenate was gently shaken, adsorbed at 37° C. for 1 h, washed with a Hank's solution, added with a medium, and then incubated in a 37° C. CO₂ incubator; a cytopathic effect (CPE) was observed, and a percentage of VeroE6 TCID₅₀ in each group was calculated, where a higher percentage meant a higher virus content (Tables 7 to 10).

TABLE 7
Percentage of VeroE6 TCID₅₀ by mice lung
tissue homogenate in 2ACE2-shRNA5 group

| | Number of | Total observed | | | |
|---|---|---|---|---|---|
| Tissue | inoculated cells | results | | | |
| homogenate | (4 wells × | Normal | Lesion | Infection | Infection |
| dilution | 10 cases) | well | well | ratio | rate |
| 10¹ | 40 | 26 | 15 | 15/40 | 37.5 |
| 10² | 40 | 31 | 8 | 8/40 | 20.0 |
| 10³ | 40 | 33 | 6 | 6/40 | 15.0 |
| 10⁴ | 40 | 37 | 3 | 3/40 | 7.5 |

TABLE 8
Percentage of VeroE6 TCID₅₀ by mice lung
tissue homogenate in shRNA5 group

| | Number of | Total observed | | | |
|---|---|---|---|---|---|
| Tissue | inoculated cells | results | | | |
| homogenate | (4 wells × | Normal | Lesion | Infection | Infection |
| dilution | 10 cases) | well | well | ratio | rate |
| 10¹ | 40 | 2 | 38 | 38/40 | 95.0 |
| 10² | 40 | 5 | 35 | 35/40 | 87.5 |
| 10³ | 40 | 10 | 30 | 30/40 | 75.0 |
| 10⁴ | 40 | 16 | 24 | 24/40 | 60.0 |

TABLE 9
Percentage of VeroE6 TCID₅₀ by mice lung tissue
homogenate in positive control group

| | Number of | Total observed | | | |
|---|---|---|---|---|---|
| Tissue | inoculated cells | results | | | |
| homogenate | (4 wells × | Normal | Lesion | Infection | Infection |
| dilution | 10 cases) | well | well | ratio | rate |
| 10¹ | 40 | 2 | 38 | 39/40 | 97.5 |
| 10² | 40 | 4 | 36 | 36/40 | 90.0 |
| 10³ | 40 | 12 | 28 | 28/40 | 70.0 |
| 10⁴ | 40 | 16 | 24 | 24/40 | 60.0 |

TABLE 10
Percentage of VeroE6 TCID₅₀ by mice lung tissue
homogenate in negative control group

| | Number of | Total observed | | | |
|---|---|---|---|---|---|
| Tissue | inoculated cells | results | | | |
| homogenate | (4 wells × | Normal | Lesion | Infection | Infection |
| dilution | 10 cases) | well | well | ratio | rate |
| 10¹ | 40 | 38 | 2 | 2/40 | 5.0 |
| 10² | 40 | 38 | 2 | 2/40 | 5.0 |
| 10³ | 40 | 38 | 2 | 2/40 | 5.0 |
| 10⁴ | 40 | 39 | 1 | 1/40 | 2.5 |

(3) Effect of ACE2-Targeted Delivery

As was seen from Tables 7 to 10, percentages of VeroE6 TCID₅₀ (10¹ in each well) induced by lung homogenate in each group were as follows: 2ACE2-shRNA5 group was 37.5%, shRNA5 group was 95.0%, positive control group was 97.5%, and negative control group was 5.0%. Since RNAi mainly occurred in the cytoplasm, shRNA in the shRNA5 group was not easy to pass through the cell membrane, so as to have a little effect on the RNAi, and results were consistent with the positive control group; meanwhile, the shRNA in the 2ACE2-shRNA5 group had a better RNAi effect due to targeted delivery of the ACE2 to the target cytoplasm, and the percentage of VeroE6 TCID₅₀ was significantly different from that of the positive control group (p<0.05). A TCID₅₀ assay was conducted separately on the 2ACE2-shRNA16 and 2ACE2-shRNA21 according to the TCID₅₀ assay method above. The results showed that the 2ACE2-shRNA16 group, 2ACE2-shRNA21 group, shRNA16/21 group, positive control group, and negative control group had TCID₅₀ of 35%, 40%, 92.5%, 95%, and 7.5%, respectively, and the TCID₅₀ of experimental groups was significantly lower than that of the positive control group.

3. Detection and Functional Verification of ACE2-Ab

(1) Sample and Detection Method

The venous blood of the remaining 10 mice in each group was collected at the 2nd, 4th, and 6th weeks, a serum was separated, and sera of a same week in each group were mixed, and then stored at −20° C. for future use. The ACE2-Ab was determined by a double-antigen sandwich method according to instructions of the kit.

(2) Detection Results

It was seen from Table 11 that the ACE2-Ab in the lung tissue homogenate of mice in the 2ACE2-shRNA5 group was significantly higher than the ACE2-Ab in the control group (p<0.05), indicating that the ACE2 in the ACE2-shRNA group could stimulate the production of ACE2-Ab in mice.

TABLE 11
ACE2-Ab detection results (ng/L) of mouse
lung tissue homogenate in ACE2-shRNA group

| | | Weeks and detection results | | | |
|---|---|---|---|---|---|
| | ACE2 | 2nd | 4th | 6th | P |
| Group | molecule | week | Week | week | value |
| ACE2- | 2 | 44.15 ± | 46.12 ± | 49.64 ± | *p < 0.05 |
| shRNA | | 15.26* | 13.18* | 14.83* | |
| shRNA | None | 10.28 ± | 11.47 ± | 11.45 ± | |
| | | 4.77 | 5.88 | 5.66 | |

(3) Functional Verification of ACE2-Ab

The 2ACE2-shRNA5 group was used as an experimental group (containing ACE2-Ab), and the shRNA group was used as a control group (containing virus but not ACE2-Ab). The sera of 2nd, 4th, and 6th week in each group after ACE2-Ab detection were mixed, a mixed serum in each group were double-diluted, and 30 μl of each diluted serum was inoculated in a 96-well plate with VeroE6 growing in a single layer; experimental group was simultaneously inoculated with 30 μl of an undiluted mixed serum of the shRNA group, gently shaken and mixed, placed at 37° C. to conduct adsorption for 1 h, washed with a Hank's solution, added with a medium, and incubated at 37° C. in a CO₂ incubator; and the cytopathic effect (CPE) was observed within 1 week, and “+” indicated the normal cell growth, as shown in Table 12.

TABLE 12
Results of antiviral infection caused by ACE2-Ab
neutralization of receptor ACE2
on surface of VeroE6 cells (+/CPE)

| | Dilution of serum (ACE2-Ab) |
|---|---|
| | inhibiting virus-induced VeroE6 CPE |
| Group | 1:2 1:4 1:8 1:16 1:32 1:64 1:128 1:256 1:512 |
| ACE2-shRNA | + + + + + + CPECPECPE |
| shRNA | CPECPECPECPECPE + + + + |
| Positive control | CPECPECPECPECPECPECPECPECPE |
| Negative control | + + + + + + + + + |

As was seen from Table 12, since the negative control group was not inoculated with virus, there was no CPE in each well of VeroE6; the positive control group was inoculated with the virus without ACE2-Ab neutralization (the mixed serum in shRNA group was not diluted), such that VeroE6 in each well produced CPE; since the shRNA group had no neutralization effect of ACE2-Ab, VeroE6 did not have CPE only after a virus-containing self-serum was diluted at 1:64; though the experimental groups were also inoculated with the virus like the positive control group, but due to the neutralization effect of ACE2-Ab, the VeroE6 showed CPE only when the serum (ACE2-Ab content) was diluted at not less than 1:128. It showed that the ACE2-Ab could neutralize the viral receptor ACE2 on the surface of VeroE6 cells, thereby producing an antiviral effect.

To sum up, in the present disclosure, the shRNA of the artificial antibody plays a role of anti-variant strain; ACE2 plays a role of targeted delivery of shRNA, bivalent binding to RBD, inhibition of virus infection through RBD, protection of shRNA, and stimulation of ACE2-Ab production by hosts; and ACE2-Ab has an effect of blocking ACE2 receptors.

What is claimed is:

1. A preparation method of an artificial antibody, comprising the following step: synthesizing an artificial antibody comprising a short hairpin RNA (shRNA) region and an angiotensin-converting enzyme 2 (ACE2) region, wherein the shRNA region is used for targeted silencing of a coronavirus mRNA, and the ACE2 region is used for neutralization of a coronavirus spike protein receptor-binding domain (S1-RBD) and targeted delivery of the shRNA; the shRNA targets a conserved gene or a common gene of a coronavirus variant strain, and the ACE2 is a receptor of a coronavirus receptor-binding domain (RBD); the artificial antibody is prepared by ligating sense and antisense strands of the shRNA to an ACE2 polypeptide separately, such that the artificial antibody binds to the coronavirus S1-RBD through the ACE2 in a same way that Ig specifically binds to an antigen through Fab, to constitute an shRNA-ACE2-RBD-virus conjugate, thereby preventing virus infection through the RBD; alternatively, the shRNA is delivered to a target cell by the virus in the conjugate, resulting in an RNA interference (RNAi) effect on a virus-infected cell.

2. The preparation method of an artificial antibody according to claim 1, wherein the targeting a conserved gene refers to that an siRNA for synthesizing the shRNA is selected from a common gene of various pathogenic coronaviruses and variant strains thereof that are recorded in a database, such that a synthesized siRNA and/or shRNA conducts targeted interference on the common gene, thereby achieving a broad-spectrum anti-variant strain effect; and the common gene comprises but is not limited to an ultra-conserved gene, a conserved gene, and/or a gene spliced by conserved microsatellites.

3. The preparation method of an artificial antibody according to claim 1, wherein a process of synthesizing the shRNA comprises but is not limited to: synthesizing two complementary oligonucleotide polypeptide siRNAs of 21 nt to 25 nt and synthesizing a base sequence that serves as a spacer; ligating the two siRNAs and the base sequence into an shRNA duplex that has a loop in a middle part formed by base separation; and ligating an ACE2 polypeptide or an RBD polypeptide to each single strand of the shRNA duplex.

4. The preparation method of an artificial antibody according to claim 2, wherein a process of synthesizing the shRNA comprises but is not limited to: synthesizing two complementary oligonucleotide polypeptide siRNAs of 21 nt to 25 nt and synthesizing a base sequence that serves as a spacer; ligating the two siRNAs and the base sequence into an shRNA duplex that has a loop in a middle part formed by base separation; and ligating an ACE2 polypeptide or an RBD polypeptide to each single strand of the shRNA duplex.

5. The preparation method of an artificial antibody according to claim 1, wherein an siRNA that targets a conserved gene or a common gene of a variant strain and is used for synthesizing the shRNA comprises but is not limited to SEQ ID NO: 1 to SEQ ID NO: 39.

6. The preparation method of an artificial antibody according to claim 1, wherein the siRNA that targets a conserved gene or a common gene of a variant strain comprises but is not limited to SEQ ID NO: 5, SEQ ID NO: 7 to SEQ ID NO: 10, SEQ ID NO: 16 to SEQ ID NO: 18, SEQ ID NO: 20 to SEQ ID NO: 22, and SEQ ID NO: 30 to SEQ ID NO: 32, preferably SEQ ID NO: 5, SEQ ID NO: 16, SEQ ID NO: 21, and SEQ ID NO: 30.

7. The preparation method of an artificial antibody according to claim 5, wherein the siRNA that targets a conserved gene or a common gene of a variant strain comprises but is not limited to SEQ ID NO: 5, SEQ ID NO: 7 to SEQ ID NO: 10, SEQ ID NO: 16 to SEQ ID NO: 18, SEQ ID NO: 20 to SEQ ID NO: 22, and SEQ ID NO: 30 to SEQ ID NO: 32, preferably SEQ ID NO: 5, SEQ ID NO: 16, SEQ ID NO: 21, and SEQ ID NO: 30.

8. The preparation method of an artificial antibody according to claim 1, wherein the ACE2 is selected from but not limited to the group consisting of an extracellular ACE2 with amino acid sequences 1 to 740, a transmembrane ACE2 with amino acid sequences 741 to 763, an intracellular ACE2 with amino acid sequences 764 to 805, a full-length ACE2, and an amino acid codon-optimized ACE2 and a polypeptide thereof.

9. The preparation method of an artificial antibody according to claim 1, wherein the artificial antibody comprises but is not limited to compounds prepared by separately ligating the ACE2 to the siRNA/shRNA synthesized by SEQ ID NO: 1 to SEQ ID NO: 39, comprises but is not limited to compounds prepared by separately inserting the siRNA/shRNA synthesized by SEQ ID NO: 1 to SEQ ID NO: 39 into a middle part of the ACE2 polypeptide, and comprises but is not limited to siRNA drugs prepared by encapsulating the compounds with lipid nanoparticles.

10. The preparation method of an artificial antibody according to claim 8, wherein the artificial antibody comprises but is not limited to compounds prepared by separately ligating the ACE2 to the siRNA/shRNA synthesized by SEQ ID NO: 1 to SEQ ID NO: 39, comprises but is not limited to compounds prepared by separately inserting the siRNA/shRNA synthesized by SEQ ID NO: 1 to SEQ ID NO: 39 into a middle part of the ACE2 polypeptide, and comprises but is not limited to siRNA drugs prepared by encapsulating the compounds with lipid nanoparticles.

11. The preparation method of an artificial antibody according to claim 1, wherein the ligating or the synthesizing comprises but is not limited to ligating the ACE2 with a 3′-end of the antisense strand and a 5′-end or a 3′-end of the sense strand of the shRNA; the ligating or the synthesizing comprises but is not limited to conducting the ligating by chemical coupling or covalent coupling with a carboxyhydrazone bond, a disulfide bond, a phosphodiester bond, a phosphorodithioate bond, a thioether bond, an oxime bond, an amide bond, and a maleimide-thiol bond that have a spacer arm.

12. The preparation method of an artificial antibody according to claim 9, wherein the ligating or the synthesizing comprises but is not limited to ligating the ACE2 with a 3′-end of the antisense strand and a 5′-end or a 3′-end of the sense strand of the shRNA; the ligating or the synthesizing comprises but is not limited to conducting the ligating by chemical coupling or covalent coupling with a carboxyhydrazone bond, a disulfide bond, a phosphodiester bond, a phosphorodithioate bond, a thioether bond, an oxime bond, an amide bond, and a maleimide-thiol bond that have a spacer arm.

13. The preparation method of an artificial antibody according to claim 10, wherein the ligating or the synthesizing comprises but is not limited to ligating the ACE2 with a 3′-end of the antisense strand and a 5′-end or a 3′-end of the sense strand of the shRNA; the ligating or the synthesizing comprises but is not limited to conducting the ligating by chemical coupling or covalent coupling with a carboxyhydrazone bond, a disulfide bond, a phosphodiester bond, a phosphorodithioate bond, a thioether bond, an oxime bond, an amide bond, and a maleimide-thiol bond that have a spacer arm.

14. The preparation method of an artificial antibody according to claim 1, wherein the artificial antibody comprises but is not limited to extracellular ACE2-shRNA-extracellular ACE2, transmembrane ACE2-shRNA-transmembrane ACE2, intracellular ACE2-shRNA-intracellular ACE2, and full-length ACE2-shRNA-full-length ACE2 that are prepared by ligating an extracellular ACE2, a transmembrane ACE2, an intracellular ACE2, and a full-length ACE2 to the sense and antisense strands of the shRNA, respectively, as well as transmembrane ACE2-shRNA-extracellular ACE2 and intracellular ACE2-shRNA-extracellular ACE2 that are prepared by inserting the shRNA/siRNA into a middle part of transmembrane ACE2-extracellular ACE2 and intracellular ACE2-extracellular ACE2, respectively.

15. The preparation method of an artificial antibody according to claim 11, wherein the artificial antibody comprises but is not limited to extracellular ACE2-shRNA-extracellular ACE2, transmembrane ACE2-shRNA-transmembrane ACE2, intracellular ACE2-shRNA-intracellular ACE2, and full-length ACE2-shRNA-full-length ACE2 that are prepared by ligating an extracellular ACE2, a transmembrane ACE2, an intracellular ACE2, and a full-length ACE2 to the sense and antisense strands of the shRNA, respectively, as well as transmembrane ACE2-shRNA-extracellular ACE2 and intracellular ACE2-shRNA-extracellular ACE2 that are prepared by inserting the shRNA/siRNA into a middle part of transmembrane ACE2-extracellular ACE2 and intracellular ACE2-extracellular ACE2, respectively.

16. The preparation method of an artificial antibody according to claim 12, wherein the artificial antibody comprises but is not limited to extracellular ACE2-shRNA-extracellular ACE2, transmembrane ACE2-shRNA-transmembrane ACE2, intracellular ACE2-shRNA-intracellular ACE2, and full-length ACE2-shRNA-full-length ACE2 that are prepared by ligating an extracellular ACE2, a transmembrane ACE2, an intracellular ACE2, and a full-length ACE2 to the sense and antisense strands of the shRNA, respectively, as well as transmembrane ACE2-shRNA-extracellular ACE2 and intracellular ACE2-shRNA-extracellular ACE2 that are prepared by inserting the shRNA/siRNA into a middle part of transmembrane ACE2-extracellular ACE2 and intracellular ACE2-extracellular ACE2, respectively.

17. The preparation method of an artificial antibody according to claim 13, wherein the artificial antibody comprises but is not limited to extracellular ACE2-shRNA-extracellular ACE2, transmembrane ACE2-shRNA-transmembrane ACE2, intracellular ACE2-shRNA-intracellular ACE2, and full-length ACE2-shRNA-full-length ACE2 that are prepared by ligating an extracellular ACE2, a transmembrane ACE2, an intracellular ACE2, and a full-length ACE2 to the sense and antisense strands of the shRNA, respectively, as well as transmembrane ACE2-shRNA-extracellular ACE2 and intracellular ACE2-shRNA-extracellular ACE2 that are prepared by inserting the shRNA/siRNA into a middle part of transmembrane ACE2-extracellular ACE2 and intracellular ACE2-extracellular ACE2, respectively.

18. The preparation method of an artificial antibody according to claim 1, wherein the artificial antibody comprises but is not limited to 2ACE2-shRNA5, 2ACE2-shRNA16, 2ACE2-shRNA21, and 2ACE2-shRNA30.

19. The preparation method of an artificial antibody according to claim 14, wherein the artificial antibody comprises but is not limited to 2ACE2-shRNA5, 2ACE2-shRNA16, 2ACE2-shRNA21, and 2ACE2-shRNA30.

20. The preparation method of an artificial antibody according to claim 15, wherein the artificial antibody comprises but is not limited to 2ACE2-shRNA5, 2ACE2-shRNA16, 2ACE2-shRNA21, and 2ACE2-shRNA30.
